(19) United States
(12) Patent Application Publication
(10) Pub. No.: US 2007/0021727 A1
(43) Pub. Date: Jan. 25, 2007

(54) Disposable absorbent undergarment with pocket

(76) Inventors: Marco Antonio Fabrega, Coral Springs, FL (US); Diana Esther Fabrega, Coral Springs, FL (US); Esther Cacho, Coral Springs, FL (US); Deborah Stephens, Coral Springs, FL (US); Lascelles Stephens, Coral Springs, FL (US)

Correspondence Address: MARCO FABREGA, 4613 N. UNIVERSITY DR, #280, CORAL SPRINGS, FL, 33067, US

(21) Appl. No.: 11/161,107

(22) Filed: Jul. 22, 2005

(51) Int. Cl. A61F 13/15

(52) U.S. Cl. 604/385.19

(57) ABSTRACT

An absorbent disposable article, such as an incontinent adult article, having a pocket for the placement of moisturizing pads in the immediate interior surface thereof.

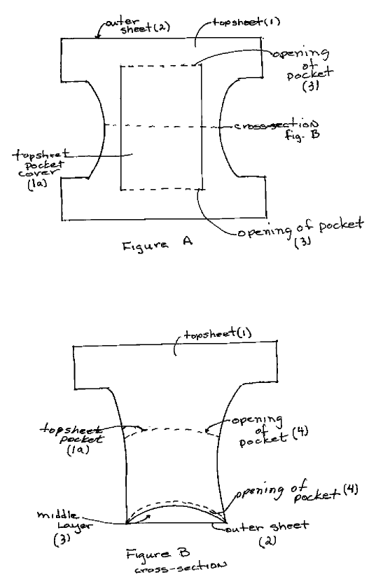

### BACKGROUND OF INVENTION

This invention generally relates to absorbent articles and, more particularly, to an improved disposable garment for post partum and incontinent women.

Currently post-partum women wear disposable feminine hygiene pads to receive and contain discharge such as blood and urine. These pads do not allow for isolation of post-partum bleeding and fluids to be isolated from the wearer's outergarments nor soiling of the wearer's surroundings.

This proposed disposable absorbent undergarment functions both to contain the discharged blood or other body fluids and to isolate these materials from the body of the wearer and the wearer's surrounding while allowing a soothing moisturizing pad to be placed in the pocket in the interior of the absorbent undergarment.

Prior art of disposable absorbent undergarment disclose three basic structural elements: a fluid permeable topsheet designed to be placed next to the wearer's skin, a backsheet which forms with the outer surface of the undergarment and an absorbent element interposed between the topsheet and the backsheet. The topsheet is permeable to fluids. The backsheet is usually liquid impermeable or repellent. Its function is to contain fluids within the absorbent element thereby protecting the wearer's outer garments or other surfaces from soiling by these fluids. Backsheets are commonly formed of a thin sheet of polyethylene, polypropylene or other flexible moisture impeding material which is substantially water impervious. The absorbent core of prior art of absorbent undergarments is comprised of a mass of hydrophilic fiber material. These fibers are often formed at least in part from cellulose. The core is intended to absorb fluids that permeate the topsheet. The core is capable of absorbing fluids many times its weight.

### BRIEF DESCRIPTION OF THE INVENTION

Generally stated, the present invention provides a distinct pocket on the topsheet of the undergarment. This will allow the wearer to place moisturizing pads in the pocket provided on the topsheet. The wearer will then have a soothing surface placed immediately to the traumatized post-partum perineal area. The pockets also will provide the availability for placement of moisturizing pads that will also provide a soothing surface for the post-partum C-section wound site. The absorbent undergarment will allow for the capture of blood and other bodily fluids.

### DESCRIPTION OF THE DRAWING

In figure A, (1.) topsheet (1a.) the topsheet pocket (2.) the outer sheet (3.) opening of pocket

In figure B (cross-section of figure A), (1.) topsheet (1a.) topsheet pocket (2.) outer sheet (3.) middle layer (4.) opening of pocket

### DETAILED DESCRIPTION OF THE INVENTION

Generally speaking, a disposable undergarment has at least three operative layers. These layers include a liquid pervious inner layer that contacts the wearer's skin, an outer liquid impervious layer on the side of the inner layer opposite the wearer's body, and an absorbent layer positioned between the inner and outer layers. The absorbent undergarment may also include upstanding leg cuffs that extend generally longitudinally along the undergarment at least in the crotch area and leg elastics that generally are positioned about the leg openings. The materials of construction, methods of construction and functionality of these various absorbent undergarment structures are well-known to those skilled in the art.

For the purpose of the present invention, only the absorbent undergarment structure in immediate contact with the wearer's skin will be discussed in detail. This structure is the inner layer. As mentioned before the inner layer is liquid pervious and generally constructed from natural or woven or nonwoven synthetic fabric which allows urine and other liquids to pass through the layer away from the wearer's skin and into the absorbent layer.

One of the objectives of the present invention is the inclusion of an additional liquid pervious layer of a natural or woven or nonwoven synthetic fabric which is approximately two thirds the length of the attached previously described inner liquid pervious layer.

This addition of a partial length liquid pervious layer will create a pocket in the crotch area of the absorbent undergarment. The pocket created can be used for placement of moisturizing pads in a fixed position. The positioning of the pads will be in such a fashion that the area provided will include the perineal region and the C-section incision site.

Having described the invention in rather full detail, it will be readily apparent to a person of ordinary skill that various changes and modifications can be made without departing from the spirit of the invention. All such changes and modifications are contemplated as being within the scope of the present invention.

What is claimed is:

1. An integral disposable absorbent article comprising:
A topsheet:
a) liquid pervious
b) a pocket two thirds the length of the topsheet centrally placed
c) attached laterally to the longitudinal edge of the absorbent article
A backsheet:
An absorptive core disposed between and attached to the topsheet and backsheet
And elasticized unitary cuff.
